SEQUENCE LISTING (11 sequences)

SEQ ID NO: 1 - DNA, 20 bp, Artificial Sequence
  primer
gggccctgaccttttcagac

SEQ ID NO: 2 - DNA, 20 bp, Artificial Sequence
  primer
ccacccctttccctttcatt

SEQ ID NO: 3 - DNA, 56 bp, Artificial Sequence
  linker oligonucleotide
  variation 35..42: /note=“N is A, C, G or T”
gtctcgtgggctcggagatgtgtataagagacagnnnnnnnncgctcttccgatct

SEQ ID NO: 4 - DNA, 26 bp, Artificial Sequence
  linker oligonucleotide
gatcggaagagcgaaaaaaaaaaaaa

SEQ ID NO: 5 - DNA, 60 bp, Artificial Sequence
  primer
  variation 34..40: /note=“N is A, C, G or T”
tcgtcggcagcgtcagatgtgtataagagacagnnnnnnngggatatttggggctcatgg

SEQ ID NO: 6 - DNA, 62 bp, Artificial Sequence
  primer
  variation 34..40: /note=“N is A, C, G or T”
tcgtcggcagcgtcagatgtgtataagagacagnnnnnnngacagcccatcctatagcactc

SEQ ID NO: 7 - DNA, 19 bp, Artificial Sequence
  primer
gtctcgtgggctcggagat

SEQ ID NO: 8 - DNA, 20 bp, Artificial Sequence
  primer
tggggcgacatctgaagaaa

SEQ ID NO: 9 - DNA, 20 bp, Artificial Sequence
  primer
tgcagggttggagtttcaga

SEQ ID NO: 10 - DNA, 20 bp, Artificial Sequence
  primer
ccttaatcacgctctggtgc

SEQ ID NO: 11 - DNA, 20 bp, Artificial Sequence
  primer
gcggacttgggccttatcat

(19) United States
(12) Patent Application Publication
(10) Pub. No.: US 2020/0123615 A1
(43) Pub. Date: Apr. 23, 2020

(54) MONITORING METHOD FOR ADULT T-CELL LEUKEMIA/LYMPHOMA (ATL)

(71) Applicants: UNIVERSITE DE LIEGE, Angleur (BE); INSTITUT JULES BORDET, Bruxelles (BE); UNIVERSITE LIBRE DE BRUXELLES, Bruxelles (BE)

(72) Inventors: Michel Georges, Liege (BE); Anne Van Den Broeke, Bruxelles (BE); Keith Durkin, Liege (BE); Maria Artesi, Liege (BE); Vincent Hahaut, Liege (BE); Nicolas Rosewick, Liege (BE)

(21) Appl. No.: 16/500,910

(22) PCT Filed: Apr. 6, 2017
(86) PCT No.: PCT/EP2017/058271

§ 371 Date: Oct. 4, 2019

(51) Int. Cl. C12Q 1/6886; C12Q 1/6806; C12Q 1/686; C12Q 1/70

(52) U.S. Cl. CPC C12Q 1/6886; C12Q 1/6806; C12Q 2600/106; C12Q 1/702; C12Q 2600/118; C12Q 1/686

(57) ABSTRACT

The present invention refers to a method for preparing a linear PCR product from genomic DNA derived from cells of a host subject infected with an retrovirus or a subject suffering from a disease associated with said retrovirus,

wherein the PCR product contains a target sequence comprising an integration site of the retrovirus in the host genomic DNA of the cells, said integration site comprising at least the terminal end of 3′-LTR or 5′-LTR sequence of the retrovirus and the adjacent host genomic DNA sequence,

wherein the PCR product comprises a first terminus and a second terminus and sequences in the following order:
sequences specific for the first terminus, a sequence comprising at least 6 consecutive random nucleotides followed by a linker sequence, host genomic DNA sequence, at least the terminal end of 3″-LTR or 5″-LTR sequence of the retrovirus, sequences specific for the second terminus;

wherein the PCR product is prepared by specific steps.

The present invention also refers to a method for determining and longitudinally monitor the dominant leukemic T lymphocyte clone in subjects suffering from Adult T-cell leukemia/lymphoma (ATL),

wherein a linear PCR product is prepared by the method according to the first aspect of the present invention, said PCR product is subjected to multiplex sequencing thereby determining all insertion sites and all shearing sites, the shearing sites are correlated to the respective insertion site,

followed by counting the number of different shear sites for each insertion site representing a specific T lymphocyte clone, removing any PCR duplicate from consideration by eliminating reads that have the same insertion site and the same random tag, and determining the abundance of each specific T lymphocyte clone therefrom.

The present invention refers to a method for preparing a linear PCR product from peripheral blood mononuclear cells (PBMCs) derived from subjects infected with HTLV-1 or subjects suffering from Adult T-cell leukemia/lymphoma (ATL), and a method for determining and longitudinally monitoring the dominant malignant/leukemic T lymphocyte clone in subjects suffering from Adult T-cell leukemia/lymphoma.

An estimated 10 to 20 million people are infected with the human T-cell leukemia Virus type-1 (HTLV-1 worldwide. In about 5% of infected individuals the virus provokes Adult T-cell leukemia/lymphoma (ATL), an aggressive CD4+T-cell malignancy with a very poor prognosis. The oncogenic retrovirus HTLV-1 has a global but uneven distribution with endemic areas in southwestern Japan, the Caribbean basin, Central and South America, West Africa and pockets in the Middle East and Europe. Although the cumulative incidence of ATL among HTLV-1 infected individuals is only 5%, it has an extremely poor prognosis and remains a major health concern in endemic regions. Patients with ATL present with diverse clinical features that include increased abnormal lymphocytes with flower-like nuclei (flower cells) in the peripheral blood, lymphadenopathy, hypercalcemia, skin lesions, multi-organ failure and/or frequent opportunistic infections. ATL has been classified into four clinical subtypes,—acute, lymphoma, smoldering and chronic—based on blood involvement, the presence of circulating abnormal lymphocytes and biological parameters that include hypercalcemia and lactate dehydrogenase (LDH) levels. This sub-classification greatly influences the treatment regimen and prognosis of the patients. Patients with an aggressive subtype have an extremely poor prognosis with a median survival of 4 to 6 months for the acute leukemic subtype and 9 to 10 months for the lymphoma subtype. Indolent forms have a more encouraging prognosis, with median overall survival of 33 and 51 months for chronic and smoldering subtypes respectively.

Treatment strategies for ATL mainly depend on clinical subtype, prognostic factors and response to initial therapy. In Japan, aggressive forms (acute, lymphoma and unfavorable chronic subtypes) are treated by conventional chemotherapy combined or not with allogeneic stem cell transplantation (allo-SCT), while in western countries the combination of interferon (IFN) alpha with zidovudine (AZT) is currently the standard first-line therapy for chronic and the majority of acute ATLs. Studies of combination therapies that include targeted treatments like Arsenic trioxide (As₂0₃) and antibody to CCR4 have shown potential clinical benefit. Response to treatment and complete clinical remission are currently defined on the basis of morphological and cytological consensus criteria i.e. the normalization of complete blood counts (CBC), the presence of <5% abnormal lymphocytes in the blood and the absence of measurable tumors for >4 weeks. Given the extremely poor prognosis, the high rates of rapid relapse and the marked diversity in survival outcome after achieving complete hematological remission, a revision of the current consensus criteria is required, with a need for improved molecular tools that integrate specific aspects of the pathophysiology of ATL/HTLV-1 and better estimate response to treatment. One of the main molecular attributes of HTLV-1 infected cells is the proviral integration site in the host genome. While HTLV-1-infected asymptomatic carriers and non-malignant HTLV-1-related diseases are characterized by a large number of clones of varying abundance, each uniquely identified by their proviral integration site in the host genome, the development of ATL is associated with the emergence of a single dominant clone, with an underlying polyclonal population of infected cells. In the majority of ATL cases examined to date the presumed malignant clone carries a single proviral integration.

Gillet et al. (The host genomic environment of the provirus determines the abundance of HTLV-1-infected T-cell clones. Blood 117:3113-3122, 2011) developed an approach based on ligation-mediated PCR and massively parallel sequencing that allows simultaneously the mapping and the quantification of the abundance of each clone.

However, the method of Gillet et al. only assays the 3′LTR, which constrains the potential dynamic range of the assay and precludes identifying clones with a deleted 5′LTR. Further, the determination of clone abundance is not optimal, and the duration of carrying out the method is high, as well as the costs.

There was a need for improved molecular tools that better estimate response to treatment. Therefore, the object of the present invention was to develop a method providing a more accurate determination of clone abundance in tumors, detection of 5′ deletions, to longitudinally monitor the malignant clone in ATL patients and better evaluate molecular response. A further object was to reduce hands-on time and the costs for the method.

The object of the present invention was solved by a method for preparing a linear PCR product from genomic DNA derived from cells of a host subject infected with a retrovirus or a subject suffering from a disease associated with said retrovirus, wherein the PCR product contains a target sequence comprising an integration site of the retrovirus in the host genomic DNA of the cells, said integration site comprising at least the terminal end of 3′-LTR or 5′-LTR sequence of the retrovirus and the adjacent host genomic DNA sequence,

wherein the PCR product comprises a first terminus and a second terminus and sequences in the following order:

sequences specific for the first terminus, a sequence comprising at least 6 consecutive random nucleotides followed by a linker sequence, host genomic DNA sequence, at least the terminal end of 3′-LTR or 5′-LTR sequence of the retrovirus, sequences specific for the second terminus;

wherein the PCR product is prepared by the following steps:

a) isolating genomic DNA from cells derived from said subject;

b) shearing the genomic DNA obtained in step a);

c) carrying out an extension reaction using the sheared DNA obtained in step b) in the presence of a primer binding to 5′-LTR of the retrovirus, a primer binding to 3′-LTR of the retrovirus, dNTPs and a DNA polymerase, wherein the dNTPs comprise labelled dNTPs and/or the primers are labelled, thereby producing a double stranded DNA product comprising a labelled DNA strand having a 3′ single nucleotide overhang; wherein the label represents a binding ligand;

d) ligating a linker having a 3′ single nucleotide overhang to the terminus of the product obtained in step c), wherein the 3′ single nucleotide overhang of the linker is complementary to the 3′ single nucleotide overhang of the labelled DNA strand;

thereby introducing the sequence comprising at least 6 consecutive random nucleotides followed by a linker sequence and at least one of the sequences specific for the first terminus,

e) isolating the product obtained in step d) using a receptor binding to the binding ligand;

f) carrying out at least one PCR reaction in the presence of the product obtained in step e) as template, using a first primer specific the first terminus and a second primer specific the second terminus of the product obtained in step e), wherein the first and the second primer each comprise at least one additional sequence different from each other at its 5′-end, thereby producing said linear PCR product comprising said additional sequence specific for the first terminus and said additional sequence specific for the second terminus.

The advantage of preparing labeled (i.e. biotinylated) DNA constructs is the selection and enrichment of viral LTR-containing fragments and increased sensitivity of the method as the labeled (biotinylated) DNA is purified in a subsequent purification step.

A further advantage of the present approach is that both 3′-LTR and 5′-LTR-host junction sequences are detected, which increases the dynamic range and the number of integration sites that can be detected. This approach therefore enables the identification of HTLV-1 variants that have a deletion of the 5′-LTR (type 2 defective HTLV-1 provirus. These variants are observed in one third of the Adult T-cell leukemia (ATL) cases. Detection of these variants are also important as it has been suggested that these ATLs have a worse prognosis compared to those that carry a full-length provirus.

The sequence comprising at least 6 consecutive random nucleotides preferably comprises 6 to 30 consecutive random nucleotides (which herein may be also termed “random tag” or “random tag sequence”). Preferably, the random tag sequence comprises 6 to 20, further preferred 6 to 10 consecutive random nucleotides. However, in a specifically preferred embodiment of the method 8 consecutive random nucleotides are used. The random tag is used to detect PCR duplicates, i.e. reads that have the same shear site and the same random tag. This approach enables more accurate determination of clonal abundance.

The random tag is followed by a linker sequence having at least 10, preferably at least 12 nucleotides, more preferred 10 to 60, further preferred 12 to 25 nucleotides.

The method according to the present invention provides a more accurate determination of clone abundance in tumors, detection of 5′ deletions, and provides the tool to longitudinally monitor the malignant clone in ATL patients and to improve evaluation molecular response. In addition, hands-on time and the costs for the method were reduced.

According to the present invention the DNA polymerase used in the extension step c) adds a single extra nucleotide to the 3′-end of the synthetized DNA strand.

In a preferred embodiment in step c) a double stranded DNA product is obtained comprising at least the terminal end of 3′-LTR or 5′-LTR sequence of the retrovirus and the adjacent host genomic DNA sequence of the insertion site as well as the shear site.

Further preferred in step d) the linker is added to that terminus of the product obtained in step c) which is opposite to the terminus carrying the 3′-LTR or 5′-LTR, respectively.

In a further preferred method in step d) a double stranded DNA comprising a labelled DNA strand is obtained, wherein the double stranded DNA product comprises at least the terminal end of 3′-LTR or 5′-LTR sequence of the retrovirus, the adjacent host genomic DNA sequence of the insertion site as well as the shear site, the sequence comprising at least 6 consecutive random nucleotides followed by the linker sequence and at least one of the sequences specific for the first terminus.

In a further preferred embodiment in step d) a linker having an overhanging single nucleotide is ligated to that terminus of the product obtained in step c) having the 3′ overhanging single nucleotide, wherein the overhanging single nucleotide of the linker hybridizes to the 3′ overhanging single nucleotide of the DNA strand synthesized in step c); thereby producing a double stranded DNA product comprising a labelled DNA strand, wherein the double stranded DNA product comprises at least the terminal end of 3′-LTR or 5′-LTR sequence of the retrovirus and the adjacent host genomic DNA sequence of the insertion site as well as the shear site, the sequence comprising at least 6 consecutive random nucleotides followed by the linker sequence and at least one of the sequences specific for the first terminus,

In another preferred embodiment of the method the overhanging single nucleotide added to the 3′-end of the synthetized DNA strand in step c) is deoxyadenosine and the overhanging single nucleotide of the linker added in step d) is desoxythymidine.

In still a further preferred embodiment of the method the PCR product comprises in the following order X4 sequence, X3 sequence, the sequence comprising at least 6 consecutive random nucleotides followed by the linker sequence, host genomic DNA sequence, at least the terminal end of 3′-LTR or 5′-LTR sequence of the retrovirus, X1 sequence, X2 sequence;

and wherein step f) comprises the following steps:

f1) carrying out a first PCR reaction in the presence of the product obtained in step e) as template, a primer binding to the X3 sequence, a primer binding to 5′-LTR, a primer binding to 3′-LTR, wherein the primer binding to 5′-LTR and 3′-LTR, respectively, comprise an additional X1 sequence at their 5′-ends, thereby producing a PCR product comprising also the X1 sequence;

f2) carrying out a second PCR reaction in the presence of the product obtained in step f1) as template, a primer binding to the X3 sequence having an additional X4 sequence at its 5′-end, a primer binding to the X1 sequence having an additional X2 sequence at its 5′-end, thereby producing a PCR product comprising also the X2 sequence and the X4 sequence.

In a preferred embodiment of the method of the present invention the X3 sequence sequence comprises Nextera Reverse sequence to which Nextera Reverse primer binds. The X3 sequence is comprised in the linker which is introduced at the ligation step. As the Nextera Reverse transposon-based sequence that is also present in Illumina Nextera Forward primers it is possible to use commercial kits for the sequencing step of the end-product libraries.

In a further preferred embodiments of the method of the present invention the X1 sequence comprises Nextera Forward sequence to which Nextera Forward primer binds, the X4 sequence comprises P7 and Index sequences and the X2 sequence comprises P5-Index sequences.

In another preferred embodiment of the method according to the present invention the binding ligand and the receptor is selected from the following group of binding pairs consisting of biotin/avidin, biotin/streptavidin; digoxygenin/anti-digoxygenin antibody; hapten/anti-hapten antibody; antigen/antibody.

In a particularly preferred embodiment of the method according to the present invention the retrovirus is HTLV-1, the HTLV-1-associated disease is Adult T-cell leukemia/lymphoma (ATL) and the genomic DNA is derived and/or purified from peripheral blood mononuclear cells (PBMCs).

The present invention also provides a method for determining and longitudinally monitor the dominant leukemic T lymphocyte clone in subjects suffering from Adult T-cell leukemia/lymphoma (ATL), wherein a linear PCR product is prepared by the method according to any one of claims 1 to 10, said PCR product is subjected to multiplex sequencing thereby determining all insertion sites and all shearing sites, the shearing sites are correlated to the respective insertion site,

followed by counting the number of different shear sites for each insertion site representing a specific T lymphocyte clone, removing any PCR duplicate from consideration by eliminating reads that have the same insertion site and the same random tag, and determining the abundance of each specific T lymphocyte clone therefrom.

The random tag is used to detect PCR duplicates, i.e. reads that have the same shear site and the same random tag. This approach enables more accurate determination of clonal abundance.

Multiplex sequencing enables the processing of a large number of samples on a high-throughput instrument. Individual “barcode” sequences are added to each sample so they can be distinguished and sorted during data analysis. Pooling samples exponentially increases the number of samples analyzed in a single run, without drastically increasing cost or time.

As mentioned above multiplex sequencing allows to determining all insertion sites and all shearing sites. This means that the insertion sites and the shearing sites of each linear PCR product is determined.

Preferably the method further comprises
judging on the basis of the abundance of each specific T lymphocyte clone the likelihood of recurrence of Adult T-cell leukemia/lymphoma (ATL); and/or
judging on the basis of the abundance of each specific T lymphocyte clone the success of treatment of Adult T-cell leukemia/lymphoma (ATL).

According to further preferred methods of the present invention

a higher abundance of a specific T lymphocyte clone indicates a higher likelihood of recurrence of Adult T-cell leukemia/lymphoma (ATL) and/or that the treatment of Adult T-cell leukemia/lymphoma (ATL) is not successful;

a lower abundance of a specific T lymphocyte clone indicates a lower likelihood of recurrence of Adult T-cell leukemia/lymphoma (ATL) and/or the that the treatment of Adult T-cell leukemia/lymphoma (ATL) is successful.

According to a further preferred method of the present invention the specific T lymphocyte clone is the same as identified as dominant leukemic clone at diagnosis. However, when monitoring the subjects during or after treatment of it may turn out that the dominant specific T lymphocyte clone is different from the dominant specific T lymphocyte clone identified at diagnosis.

The present inventors developed an improved high-throughput sequencing (HTS) methods to simultaneously map HTLV-1 insertion sites and measure the abundance of the corresponding clones means that it is now possible to monitor ATL patients, both on and off therapy, based on the evolution of the predominant malignant clone identified at diagnosis.

With this method according to the present invention it was possible to analyze HTLV-1 clonality in retrospective longitudinal samples of 5 patients with ATL who all achieved complete hematological remission, yet relapsed after variable courses/periods of clinical response.

### DETAILED DESCRIPTION

The invention is described in more detail by the following figures.

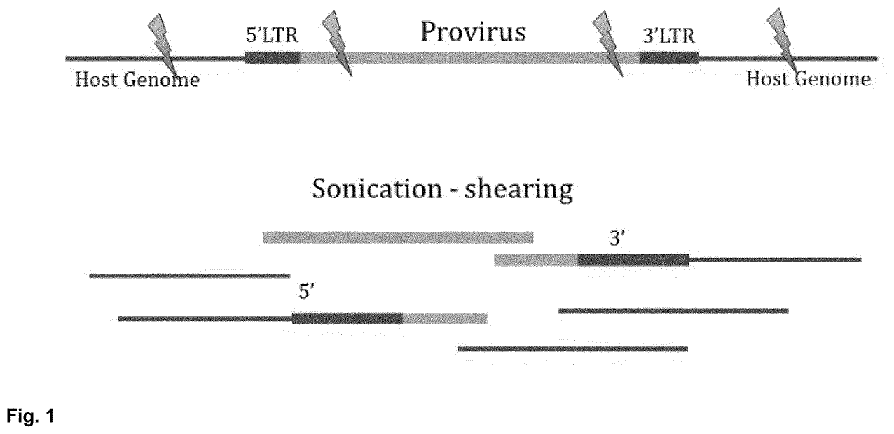

FIG. 1 shows a schematic representation of the provirus integrated into the host genome and possible fragments obtained after DNA shearing.

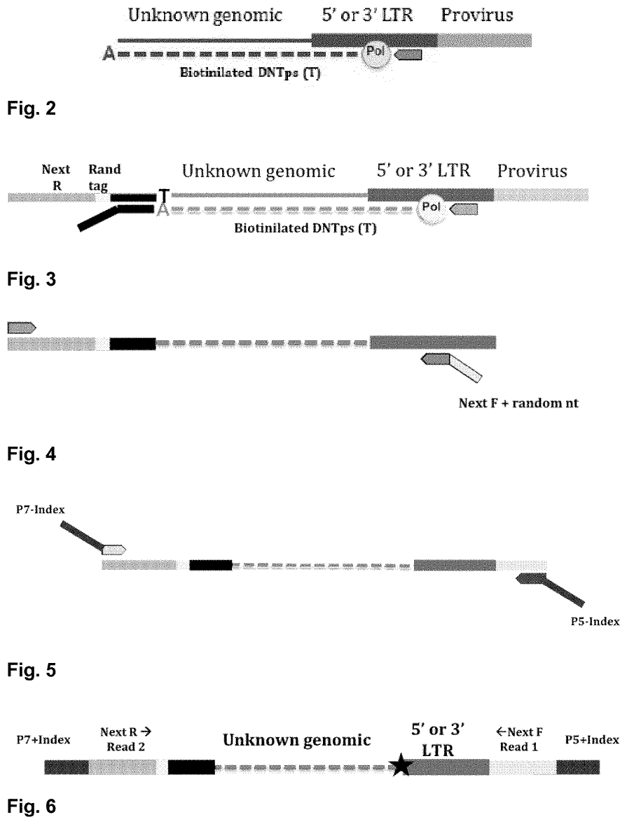

FIG. 2 shows the DNA extension step by example of a DNA fragment containing a part of the proviral sequence.

FIG. 3 shows the ligation step.

FIG. 4 shows the first PCR step (PCR 1).

FIG. 5 shows the second PCR step (PCR 2).

FIG. 6 points out the junction between proviral and host genome sequences.

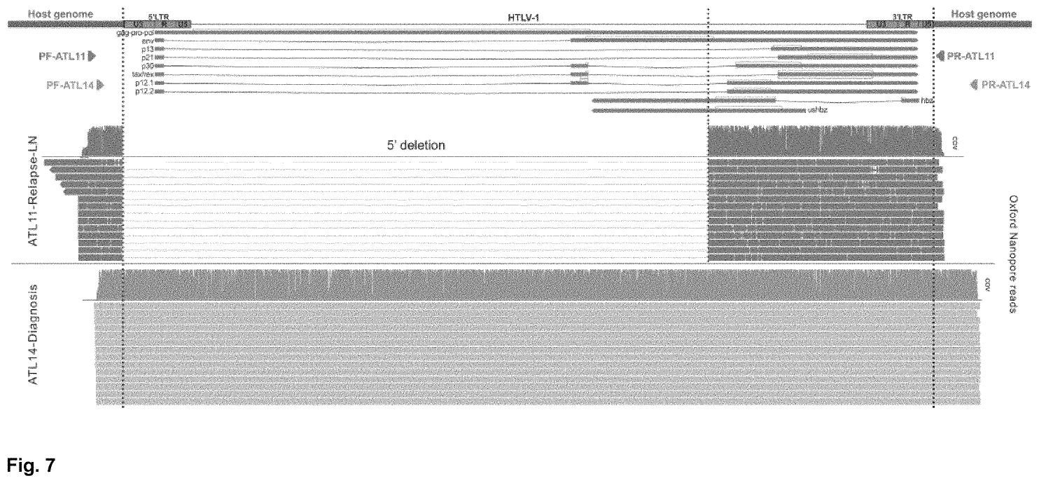

FIG. 7 shows the longitudinal monitoring of the HTLV-1 dominant malignant clone and associated clone frequency distribution in 5 ATL patients.

FIG. 1 shows a schematic representation of the provirus integrated into the host genome and possible fragments obtained after DNA shearing. The viral genome of the HTLV-1 is flanked by viral 5′-LTR and 3′-LTR sequences. There are also integrated proviruses where the 5′-LTR and adjacent viral sequences are deleted (not shown). After shearing the genomic DNA diverse fragments can be obtained. DNA fragments containing only host genomic sequences, DNA fragments containing only proviral sequences, DNA fragments containing host genomic sequences and parts of viral sequences.

FIG. 2 shows the DNA extension step by example of a DNA fragment containing a part of the proviral sequence. The extension step makes use of both primers specifically binding to 5′-LTR and primers binding specifically binding to 3′-LTR. In the extension reaction biotinylated dNTPs are used for labelling the DNA strand to be synthesized starting from said specific primers. Alternatively, the biotin-label may be incorporated into said primers. For DNA synthesis a DNA polymerase is used which adds a single additional nucleotide to the 3′-end of the newly synthesizes (biotinylated) DNA strand, i.e. an A (adenosine desoxyribonucleotide). In this step only those DNA fragments are considered by the reaction and therefore are biotinylated which contain the 5′-LTR and 3′-LTR sequences. The advantage of this approach is the selection and enrichment of viral LTR-containing fragments and increased sensitivity. In a subsequent purification step using a streptavin-based selection these biotinylated constructs can be purified. The extension reaction also provides a sticky end at the terminus opposite to the LTR sequence where the primer binds. The sticky end is used for adding a linker only to that one terminus in the subsequent step (see FIG. 3).

A further advantage of the present approach is that both 3′-LTR and 5′-LTR-host junction sequences are detected, which increases the dynamic range and the number of integration sites that can be detected. This approach therefore enables the identification of HTLV-1 variants that have a deletion of the 5′-LTR (type 2 defective HTLV-1 provirus. These variants are observed in one third of the Adult T-cell leukemia (ATL) cases. Detection of these variants are also important as it has been suggested that these ATLs have a worse prognosis compared to those that carry a full-length provirus.

FIG. 3 shows the ligation step, where a linker is added to the sticky end at the terminus opposite to the LTR sequence. For this oligonucleotides are used which provide a partially double-stranded DNA having a 3′ single nucleotide overhang, wherein this single nucleotide of the linker is complementary to the single nucleotide added to the labelled (biotinylated) DNA strand newly synthesized by the polymerase during the extension step. As the latter is an A the 3′ single nucleotide overhang of the linker is a T (thymidine desoxyribonucleotide). The extension step and subsequent ligation step provides a DNA construct of interest which has a defined architecture: the terminus comprising the host genomic DNA is provided with a specific sequence, whereas the opposite terminus comprises the terminal end of the 3′-LTR or 5′-LTR. The DNA construct further comprises the original junction between host genomic DNA sequence and the proviral sequence (insertion site). The specific insertion site is indicative for a specific T-cell clone. The DNA construct also comprises the shear site of the genomic DNA (created during the shearing step) to which the linker is added. The shear site is being used during the evaluation for estimating the abundance of a specific clone, the latter being represented by a specific or unique insertion site as mentioned above.

In the ligation step a sequence comprising at least 6 consecutive random nucleotides followed by a linker sequence and at least one of the sequences specific for the first terminus. In the embodiment of the method described in the examples the ligation step introduces the Nextera Reverse transposon-based sequence as sequence specific for the first terminus (herein also designated as “X3” sequence) that is also present in Illumina Nextera Forward primers and thus enables the use of commercial kits for the sequencing step of the end-product libraries. Further, random tags are introduced with the linker that improve to measure clonal abundance.

FIG. 4 shows the first PCR step (PCR 1). In this first PCR step according to one embodiment of the present invention a transposon-based sequence that is also present in Illumina Nextera Forward primers is introduced and thus enables the use of commercial kits for the sequencing step of the end-product libraries. Here the first PCR reaction is carried out in the presence of the purified product obtained after extension and ligation as template, a primer binding to the linker sequence, a primer binding to 5′-LTR and a primer binding to 3′-LTR. These primer binding to 5′-LTR and 3′-LTR, respectively, here comprise an additional Illumina Nextera Forward sequence and an additional random nucleotide sequence at their 5′-ends. The PCR product therefore comprises said additional sequences.

FIG. 5 shows the second PCR step (PCR 2). In this second PCR step according to one embodiment of the present invention off-the-shelf indexing primers (Illumina) are introduced for subsequent multiplex sequencing. Here the second PCR reaction is carried out in the presence of the purified PCR product obtained after the first PCR step as template, a primer binding to the linker sequence, and a primer binding to Illumina Nextera Forward sequence (distal to the LTR sequence). Here the primer binding to the linker sequence comprises an additional P7 and -Index sequence at its 5′-end, and the primer binding to the LTR terminus comprises an additional P5 and Index sequence at its 5′-end. The second PCR provides a PCR product comprising the P7 and Index at the terminus comprising the linker and the host genomic sequence and the P5 and Index at the terminus comprising the LTR of the provirus.

Therefore, this reaction adds sequences that have P5 and P7 sequences and indexes (thus P5+index and P7+index). P5 and P7 are used to bind the flow cell, while indexes are used for multiplex sequencing.

Multiplex sequencing enables the processing of a large number of samples on a high-throughput instrument. Individual “barcode” sequences are added to each sample so they can be distinguished and sorted during data analysis. Pooling samples exponentially increases the number of samples analyzed in a single run, without drastically increasing cost or time: thus reduces cost per sample.

FIG. 6 points out the junction (asterisk) between proviral and host genome sequences. This junction representing the original insertion site is identified via high throughput paired end sequencing. The Illumina read 1 originates from the Nextera Forward, sequencing part of the 5′ or 3′ LTR as well as the proviral insertion site in the host genome. The read 2 sequence originates in the Nextera Reverse, sequencing the random tag, which allows for the identification of PCR duplicates and the shear site reuse. Following the random tag the host DNA represents the point of DNA shearing. The combined host sequences from read 1 and 2 facilitate accurate identification of the proviral integration site, identifying the clone and determining its abundance.

FIG. 7 shows the longitudinal monitoring of the HTLV-1 dominant malignant clone and associated clone frequency distribution in 5 ATL patients. Panels a-e show the evolution of the abundance of the dominant clone relative to all other infected cells is represented by longitudinal charts with distinct shapes corresponding to different time points (dot: diagnosis, square: relapse blood, triangle: relapse lymphoma, diamond: complete remission CR1, CR2 and CR3). Grey area indicates the period of complete clinical remission (Table 1). Samples for which a clonally rearranged T-cell receptor-gamma (TCR-γ) gene was detected have shapes marked with a black contour (TCR+). Clone frequency distribution is represented by pie-charts, each slice representing an independent integration site and its corresponding clonal abundance. The single dominant clone (abundance per 100 proviral copies, indicated below the pie-chart) is depicted in black. ATL60 shows evidence of 4 equally frequent proviruses in a single malignant clone (single TCR-γ rearrangement, ATL60, Table 2). The remaining underlying clones are depicted in grey. PVL: proviral load in PBMCs (tax copies per 100 cells). Absolute abundance of the malignant clone (percentage dominant HTLV-1 insertion sites in PBMCs) can be quantified from the PVL and the leukemic clone's relative abundance. Absolute abundance of malignant integration sites at complete remission ATL14-CR1<0.007% (scheme d, PVL of 0.016%, relative abundance of 43%).

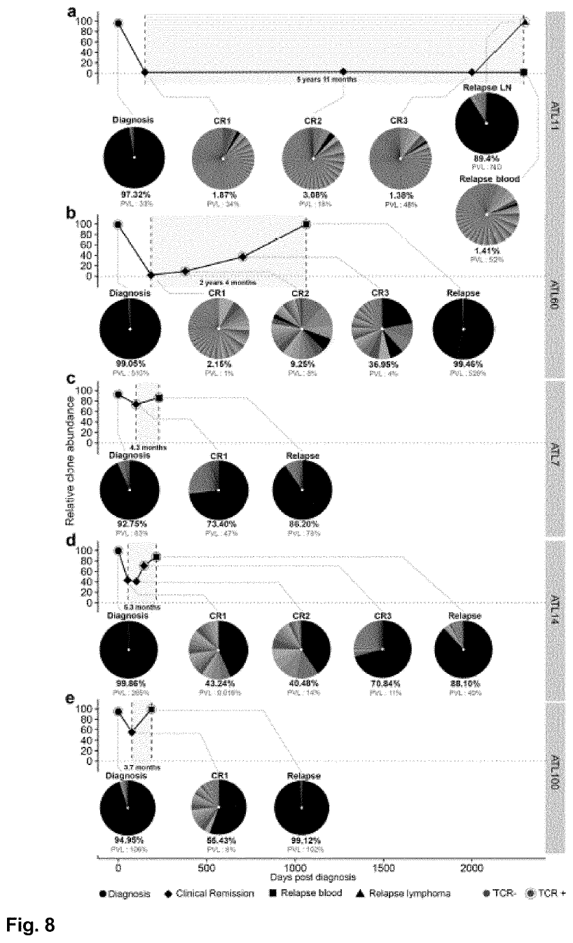

FIG. 8 shows the long-range Oxford Nanopore sequencing validates 5′/3′ dual HTS clonality method in revealing HTLV-1 type-2 defective proviruses. Coverage and individual reads generated by Oxford Nanopore sequencing of PCR products obtained with primer pairs located up- (FP) and downstream (RP) of the predominant HTLV-1 integration site in ATL11-Relapse-LN (top) or ATL14 Diagnosis (bottom) visualized in Integrative Genome Viewer (IGV) and mapped to custom genomes that have the provirus integrated into chr 1: 20,516,805 (ATL11-Relapse-LN) or chr 18: 45,011,572 (ATL14 D) in the human genome. Integration sites were identified by HTS clonality mapping. Reads spanning both human and proviral genome were on average 2,143 bp in length for ATL11-R-LN (range 41 bp to 9,155 bp) and 3,255 bp for ATL14 (range: 73 bp to 9,562 bp). ATL11-relapse-LN coverage uncovers a large 5′ deletion of 6,529 bp in the proviral genome (includes 5′LTR), consistent with the absence of 5′LTR-host reads observed with the HTS clonality method while 3′LTR-host junctions were detected. Long reads validate 5′/3′ dual HTS method for identifying type-2 defective HTLV-1 proviruses in ATL. HTLV-1 proviral genome and transcripts shown on top. PF: primer forward, PR: primer reverse, coy: coverage.

Ligand Receptor Pairs

As used herein, the phrase “ligand-receptor pair” or “binding pairs” refers to a ligand and receptor that are chemical moieties capable of recognizing and binding to each other. The ligand and receptor can be any moieties that are capable of recognizing and binding to each other to form a complex. Additionally, the ligand and receptor may interact via the binding of a third intermediary substance. Typically, the ligand and receptor constituting the ligand-receptor pair are binding molecules that undergo a specific noncovalent binding interaction with each other. The ligand and receptor can be naturally occurring or artificially produced, and optionally may be aggregated with other species.

Examples of ligands and/or receptors include, but are not limited to, agonists and antagonists for cell membrane receptors, toxins and venoms, viral epitopes, hormones such as steroids, hormone receptors, peptides, enzymes and other catalytic polypeptides, enzyme substrates, cofactors, drugs including small organic molecule drugs, opiates, opiate receptors, lectins, sugars, saccharides including polysaccharides, proteins, and antibodies including monoclonal antibodies and synthetic antibody fragments, cells, cell membranes and moieties therein including cell membrane receptors, and organelles. Examples of ligand-receptor pairs include antibody-antigen; lectin-carbohydrate; peptide-cell membrane receptor; protein A-antibody; hapten-antihapten; digoxigenin-anti-digoxigenin; enzyme-cofactor and enzyme-substrate.

In one preferred embodiment, the ligand-receptor pair is biotin-avidin or biotin-streptavidin. The vitamin biotin is detected by binding of the indicator protein avidin, isolated from egg white, or streptavidin, isolated from Streptomyces avidinii bacteria. Avidin and streptavidin have four high affinity binding sites for biotin with a binding constant of about K=10¹⁵ mol⁻.

HTS Mapping of HTLV-1 Integration Sites and Measure of Clonal Abundance

The present invention provides an improved HTS-based method which was utilized to simultaneously map and quantify the abundance of HTLV-1 integration sites in genomic DNA isolated from the patients' longitudinal PBMC samples. In addition to the random tags described in the Tag-NGS protocol developed earlier, the optimized method of the present invention includes several critical modifications in library preparation and data analysis, overcoming some of the limitations of previously established protocols. Briefly, the dynamic range of the technique was increased by assaying both the 5′LTR and 3′LTR, allowing better determination of clone abundance and providing critical information on the occurrence of 5′-deletions in the provirus. An extension step with Biotin-11-dUTP simultaneously end-repairs and facilitates streptavidin-based enrichment of LTR-positive fragments, followed by limited PCR to avoid PCR duplicates. A separate end-repair step therefore can be omitted. Off-the—shelf Illumina primers replaced custom sequencing primers for the addition of adapters and indexes, simplifying library multiplexing and reducing both the cost and hands-on time to the point where the protocol could be applied to a clinical setting. Libraries were assembled and 150-bp paired-end sequencing reads were acquired on an Illumina MiSeq instrument (mean number of raw reads: 373,400, range: 28,930-977,000). Reads that supported either 5′ or 3′LTR-host junctions were retained.

The number of unique HTLV-1 integration sites and the abundance of the corresponding clones were determined as follows: Paired-end reads were aligned to a host-provirus hybrid genome using BWA. After quality trimming (average base quality≥30) only paired-end reads that fulfilled the following conditions (spanning LTR-host junctions) were retained: Read 1: HTLV-1 5′LTR: 29 nts, HTLV-1 3′LTR: 45 nts of the read mapped to the relevant LTR extremity. Read 2: the read mapped to the host genome with 3 mismatches. Duplicates were removed based on reads that showed the same genomic insertion site and identical 8 random nt tags. Read numbers were counted for each proviral integration site and reported. Clone abundance in tumor (ATL) samples was determined as follows: if both 5′LTR and 3′LTR flanking sites were identified, %=average 3′LTR-5′LTR while if only one LTR flanking site was detected: %=% defined by detected LTR flanking site. If only 3′LTR flanking site was identified as the dominant insertion site then the clone was defined as a 51TR-deleted type-2 defective provirus.

Applying the method of the present invention, HTS-clonality revealed cases of refractoriness to first-line therapy at time points where consensus response criteria indicated complete hematological remission. In patients who achieved molecular remission, it was possible to detect states of early recurrence of the leukemic clone that escape detection by conventional methods, enabling a better prediction of relapse. HTS identified HTLV-1 variants with prognostic value, revealed clonal switch upon progression and outperformed other currently available molecular methods.

The studies of the present invention show that molecular knowledge with regards to the HTLV-1 clonal architecture in ATL patients enables a better definition of complete remission and therefore may guide therapeutic interventions. First, it was demonstrated that HTS clonality analysis can reveal ATLs refractory to first-line therapy in patients that otherwise fulfil the consensus criteria of complete hematological remission. Consistent with the persistence of the dominant malignant clone, these patients relapsed within a very short time frame. As a consequence, the development of this optimized HTS method provides clinicians with a tool to rapidly identify patients who do not benefit from standard therapy (refractory disease) and who should be enrolled in alternative or novel upfront strategies. Secondly, it was shown that in ATL patients who achieved molecular remission at one point, longitudinal molecular monitoring can detect early recurrence of the leukemic clone that escape detection by conventional methods, enabling a better prediction of relapse. Finally, HTS mapping may influence the decision to maintain treatment. In patients who achieve complete hematological remission upon AZT/IFN-alpha treatment and additionally show a sustained molecular response (defined by an HTLV-1 polyclonal architecture), the duration of maintenance therapy may be reevaluated with the potential benefit of decreasing toxicity. In addition, HTS clonality may assist in predicting the success rate for patients who qualify for allo-SCT and help tailoring immunosuppressive drugs after allo-SCT.

The high rate of relapse in ATL has been attributed to the persistence or recurrence of the predominant leukemic clone. However, some patients relapse with a different clone. HTS mapping of the ATL11 lymphoma relapse provided direct molecular evidence of such a clone switch. Identifying clonal transition at relapse may have important therapeutic implications given that treatment re-challenge may now be effective in decreasing the abundance of a novel distinct malignant clone. That the 5′/3′-dual HTS method enables the faithful identification of malignant clones that carry a 5′LTR-deleted provirus was demonstrated in the same patient and confirmed by long-range Nanopore sequencing. Integration of 5′LTR-deleted type-2 defective provirus into ATL cells is observed in approximately one third of the cases and is associated with prognosis.

Finally, while ALC and blood smears remained unremarkable over the period of complete remission in ATL14 (CR1 to CR3, absence of flower cells and ALC range 0.2 to 1.2 G/I), longitudinal HTS follow-up of this patient revealed molecular recurrence of the malignant clone at these time points (abundance of 43.24% to 70.84% in CR1 and CR3 respectively). Interestingly this patient relapsed with strong lymph node involvement (LN++) while blood parameters at relapse remained in the normal range (ALC: 1.9 G/L) (Table 2). This demonstrates that HTS analysis of the blood of a patient in complete hematological remission can predict relapse at distant sites.

This study of 5 patients provides a proof-of-concept for the integration of the HTLV-1 clonal molecular signature into response assessment criteria for the management of ATL. The HTS-based method of the present invention includes several critical modifications that overcome some of the limitations of previously reported methods and reduce both the cost and hands-on time to the point where it could be applied to a clinical trial setting.

The observations made during these studies highlight the great molecular heterogeneity within patients that achieve complete clinical and hematological remission, underlining the need for revisiting response criteria for ATL. Given the superiority of the HTS approach to any other method examined thus far, it should be integrated into consensus criteria and evaluated as a tool to better estimate response to treatment, predict relapse, and guide therapeutic interventions in the course of treatment. The present inventors propose this HTS approach as a method to detect minimal residual disease, estimate graft-versus-ATL effect after allo-SCT and evaluate clinical trials that remain critical to improving outcomes.

The present invention is described in more detail in the following examples which are not considered to limit the present invention in any way.

### EXAMPLES

Patients

The present inventors performed a longitudinal analysis of retrospective samples of five ATLs diagnosed with a leukemic subtype which all achieved clinical remission upon induction therapy yet relapsed after variable periods of hematological response. Serial samples were examined by an improved HTS-method to map proviral integration sites, enabling the molecular follow-up of the dominant leukemic clone identified at diagnosis.

In detail, the study according to the present invention was conducted on retrospective longitudinal samples of 5 ATL patients diagnosed with an aggressive leukemic type, treated at the Necker Hospital (Paris) between 2008 and 2016, and for which serial samples during clinical remission and at relapse had been archived (Table 1). The study was approved by the ethics committee CPP Ile-de-France II and all patients gave written informed consent if not deceased. Diagnosis of ATL was based on clinical parameters, the presence of atypical lymphocytes in blood smears and the presence of HTLV-1-specific antibodies in serum. Patients were classified according to the Shimoyama classification. Induction therapy consisted of either a CHOP-based regimen or AZT+IFN-alpha combination therapy. As₂0₃ was used as consolidation therapy in 2 cases. Patients' characteristics are summarized in Table 1. Complete hematological remission was defined by morphological and cytological criteria according to the recommendations published in 2009 (Tsukasaki K et al.: Definition, prognostic factors, treatment, and response criteria of adult T-cell leukemia-lymphoma: a proposal from an international consensus meeting. J Clin Oncol 27:453-9, 2009), i.e. the normalization of CBC, the presence of <5% abnormal lymphocytes in the blood and the absence of measurable tumors for >4 weeks. Patients' CBC, absolute lymphocyte counts (ALC), blood smears, clinical data and biological parameters determined by European standard protocols in the accredited diagnostic laboratory of Onco-hematology were retrospectively obtained from electronic medical records at the Necker Hospital (Table 2).

TABLE 1
Patients' characteristics

| | | | | | | Time |
|---|---|---|---|---|---|---|
| | | | | | | between |
| | | | | | | diagnosis |
| | Sex & | | | | | and |
| | Age | Ethnic | Subtype at | | | relapse |
| Patient | (years) | origin | diagnosis | Treatment | CR | (months) |
| ATL7 | F/59 | Caribbean | Acute | CHOP | 4.3 | 7.6 |
| | | | | regimen | | |
| ATL11 | M/27 | French | Unfavorable | LSG 15 | 70.7 | 75.7* |
| | | Guyana | chronic | AZT-IFN | | |
| | | | | alpha-AS₂O₃ | | |
| ATL14 | F/35 | Haiti | Acute | CHOP | 5.3 | 7.2 |
| | | | | regimen | | |
| | | | | (induction) | | |
| | | | | AZT-IFN | | |
| | | | | alpha-AS₂O₃ | | |
| | | | | (consolidation) | | |
| ATL60 | M/42 | Africa | Acute | CHOP like | 28 | 34 |
| | | | | regimen | | |
| ATL100 | F/55 | Africa | Acute | AZT-IFN alpha | 3.7 | 6.3 |

F: female,
M: male, Subtype defined according to Shimoyama classification,
CHOP: Cyclophosphamide, Adriamycin, Vincristine, Prednisone,
LSG 15: VCAP-AMP-VEPC,
IFN alpha: Interferon alpha,
AS₂O₃: Arsenic trioxide,
CR: complete remission (given in months),
*ATL11 diagnosis corresponds to the earliest available sample from this patient (non-responder clinical status after chemotherapy, prior to AZT-IFN alpha-AS₂O₃ treatment).

TABLE 2
Patients' clinical and biological characteristics

| | | ALC | Blood | ATL | Calcemia | | | | |
|---|---|---|---|---|---|---|---|---|---|
| Patient | Status | (G/I) | smear | localisation | (mmol/L) | LDH | TCR | FCM % | PVL % |
| ATL7 | D | 43.9 | >5% | Blood. LN | 2.38 | 4N | + | >80 | 83 |
| | CR1 | 1.3 | — | | 2.21 | 1.5N | + | 25 | 47 |
| | R | 3.5 | >5% | Blood. CNS | 2.26 | 7N | + | 50 | 78 |
| ATL11 | D | 1.3 | >5% | Blood | 2.27 | <1N | + | 40 | 33 |
| | CR1 | 2.1 | <5% | | 2.31 | 1.5N | − | 4 | 24 |
| | CR2 | 1.4 | — | | 2.32 | <1N | − | <5 | 18 |
| | CR3 | 1.6 | — | | ND | <1N | − | 4 | 48 |
| | R | 1.8 | <5% | LN | 2.49 | 2N | − | 6 | 52 |
| | | | | lymphoma | | | +^{#} | | |
| ATL14 | D | 16.4 | >5% | Blood. LN | 2.17 | 17N | + | 85 | 265 |
| | CR1 | 0.2 | — | | 2.3 | 6N | − | 0.20 | 0.016 |
| | CR2 | 0.6 | — | | 2.4 | 1.5N | − | 6 | 14 |
| | CR3 | 1.2 | — | | 2.46 | 1.5N | + | 10 | 11 |
| | R | 1.9 | >5% | Blood. | 2.5 | ND | + | 14 | 40 |
| | | | | LN++ | | | | | |
| ATL60 | D | 7.8 | >5% | Blood. LN | 2.97 | 5N | + | >50 | 510 |
| | CR1 | 1.3 | — | | 2.36 | 2N | − | <1 | 1 |
| | CR2 | 2.8 | — | | 2.31 | 1.5N | + | 4 | 8 |
| | CR3 | 2.6 | — | | Normal | 1.5N | + | 6 | 4 |
| | R | 47.2 | >5% | Blood | 3.7 | 6N | + | 78 | 526 |
| ATL | D | 8.0 | >5% | Blood. liver | Normal | 6N | + | >70* | 106 |
| 100 | | | | | | | | | |
| | CR1 | 3.2 | — | | Normal | 1.5N | − | 3* | 8 |
| | R | 12.4 | >5% | Blood. liver | Normal | 5N | + | >95* | 102 |

D diagnosis,
CR complete remission,
R relapse,
ALC absolute lymphocyte count,
Blood smear presence of flower cells (%),
LHD lactate dehydrogenase,
N = normal values,
TCR, clonal TCR-γ rearrangement in the blood,
^{#}TCR-γ rearrangement in lymphoma, different clone,
FCM flow cytometry immuno-phenotyping, percentage CD4⁺, CD25⁺, HLA DR⁺, CD7⁻, CD3^{dim} in PBMCs,
*ATL100 characterized by CD4⁺, CD25⁺, HLA DR⁺, CD7⁻, CD3^{high} cell population,
PVL proviral load in copies of tax per 100 PBMCs,
ND not available,
ATL11 D: corresponds to the earliest available sample from this patient (non-responder clinical status after chemotherapy, prior to AZT-IFN alpha-As₂0₃ treatment).

### Example 1: Blood Samples and DNA Preparation

Samples from HTLV-1 infected individuals were collected after informed consent obtained in accordance with the Declaration of Helsinki and after institutional review board-approved protocol at the Necker Hospital, University of Paris, France in accordance with the “Comite d'ethique Ile de France II”. Samples consisted of PBMCs from 5 ATL patients (four acute and one chronic unfavourable subtype) that underwent therapy yet relapsed. PBMCs were isolated from blood using Histopaque-1077 (Sigma). DNA used in this study was isolated using Qiagen AllPrep-DNA kit.

### Example 2: DNA Shearing

A total of 5 pg of DNA was sheared by sonication with a Bioruptor0 instrument (Diagenode, Belgium). The shearing settings were: Time ON 05 seconds—time OFF 90 seconds with a total of 8 cycles. For optimal shearing the tubes were spun down after 4 cycles. The procedure has also been performed with DNA quantities lower than 5 pg (down to 200 ng).

### Example 3: DNA Extension

The DNA obtained in Example 2 was subjected to an DNA extension step using HTLV-1 5′-LTR and 3′-LTR primers (5′-GGGCCCTGACCTTTTCAGAC-3′: SEQ ID NO: 1; 5′-CCACCCCTTTCCCTTTCATT-3′: SEQ ID NO: 2) in the presence of biotinylated dUTP (0.25 mM) (Thermo Scientific), dNTPs (dATP, dCTP, dGTP each 1 mM, dTTP 0.75 mM) and 2.5 units Hot Start Taq Polymerase (Promega). The extension program was carried out at step 1: 95° C. for 2.5 min, step 2: 95° C. for 0.5 min, step 3: 58° C. for 0.5 min, step 4: 72° C. for 1.0 min, step 5: 72° C. for 5 min; step 6: 12° C.: end of reaction. Steps 3-4 were repeated for 30 cycles. The Taq polymerase synthesizes a biotinylated DNA strand starting from the LTR-region of the provirus (where the primer binds) in the direction of the host genomic sequence and adds an additional single adenosine nucleotide to the 3′-end of this new strand.

### Example 4: Purification

The DNA obtained in Example 3 was purified by using the Agencourt Ampure XP Beads (Beckman Coulter). The products of extension were added to resuspended Agencourt Ampure XP beads in 1:1 proportions.

### Example 5: Linker Ligation

The DNA obtained in Example 4 was subjected to a ligation step, where a linker composed of oligonucleotides SEQ ID NO: 3 (5′-GTCTCGTGGGCTCGGAGATGTGTATAAGAGACAGNNNNNNNNCGCTCTTCCGA TCT-3′) and SEQ ID NO: 4 (5′-/5Phos/GATCGGAAGAGCGAAAAAAAAAAAAA-3′) was added and ligated to the DNA, thereby introducing a sequence also present in Illumina Nextera Reverse primer and a 8 nt random tag. Reaction was carried out using T4 DNA Ligase enzyme (New England Biolabs). Reactions could be carried both at room temperature for 3 hours or at 16° C. overnight.

### Example 6: Streptavidin-Based Selection

The end product of the ligation step contains biotin incorporated during the DNA extension step. The product obtained in Example 5 was selected and purified by using streptavidin beads (Dynabeads M-280, Invitrogen/Life Technologies).

### Example 7: PCR 1 Reaction

The product obtained in Example 6 was subjected to a PCR step using HTLV-1 specific primers (HTLV-5′-end-PCR primer: 5′-TCGTCGGCAGCGTCAGATGTGTATAAGAGACAGNNNNNNNGGGATATTTGGGG CTCATGG-3′: SEQ ID NO: 5; and HTLV-3′-end-PCR primer: 5′-TCGTCGGCAGCGTCAGATGTGTATAAGAGACAGNNNNNNNGACAGCCCATCCT ATAGCACTC-3′: SEQ ID NO: 6) and Linker specific primer (5′-GTCTCGTGGGCTCGGAGAT-3′ SEQ ID NO: 7) in the presence of dNTPs and Q5 Hot Start High-Fidelity Taq Polymerase (NEB) using the PCR 1 program (15 cycles): step 1: 98° C. for 30 s, step 2: 98° C. for 8 s, step 3: 66° C. for 20 s, step 4: 72° C. for 30 s, step 5: 72° C. for 60 s, step 6: 12° C.—end of reaction. Steps 2-4 are repeated for 15 cycles. This step introduces a sequence also present in Illumina Nextera Forward primer.

### Example 8: Purification

The DNA obtained in Example 7 was purified by using the Agencourt Ampure XP Beads (Beckman Coulter). The products of PCR1 were added to resuspended Agencourt Ampure XP beads in 1:1 proportions.

### Example 9: PCR 2 Reaction

The product obtained in Example 8 was subjected to a second PCR step using primers matching the Nextera primer sequences (provided by Illumina) in the presence of dNTPs and Q5 Hot Start High-Fidelity Taq Polymerase (NEB) using the PCR 1 program (12 cycles): step 1: 98° C. for 30 s, step 2: 98° C. for 8 s, step 3: 62° C. for 20 s, step 4: 72° C. for 30 s, step 5: 72° C. for 60 s, step 6: 12° C.—end of reaction. Steps 2-4 are repeated for 12 cycles. This step introduces P5 and index and P7 and index sequences.

### Example 10: Purification

The DNA obtained in Example 8 was purified by using the Agencourt Ampure XP Beads (Beckman Coulter). The products of PCR2 were added to resuspended Agencourt Ampure XP beads in 1:0.8 proportions.

### Example 11: Library Assembly

A library was constructed by pooling the different PCR 2 products (each one possessing a unique set of indexes).

### Example 12: HTLV-1 proviral Load Quantification

DNA was isolated using the Qiagen AllPrep DNA/RNA/miRNA kit and proviral DNA was quantified by real-time PCR using primers targeting HTLV-1 3′ region and RPS9 of Actin respectively for normalization (primers from Integrated DNA Technologies). Runs were performed in a 50 μL volume containing 1 μg of total DNA, primers and probe (200 nM concentration of each) in 1× PCR buffer (Platinum Quantitative PCR SuperMix-UDG) (HTLV-1) or 10 μL containing 50 ng DNA and 1×Universal PCR Master Mix, No AmpErase UNGa (ThermoScientific) (BLV). Thermocycling conditions were 10 min at 95° C., followed by 50 cycles at 95° C. for 15 sec and 60° C. for 1 min. Standard curves were generated using serial dilutions of DNA from the Tarl2 cell line (HTLV-1, single proviral copy). Proviral load in % PBMCs=(Sample Average Quantity)×2/(Sample RPS9 or Actin quantity)*100.

### Example 13: Long-Range Oxford Nanopore Sequencing of Clinical Samples

HTLV-1 proviruses and their respective 5′ and 3′flanking genomic sequences were PCR-amplified using LongAmp® Taq DNA Polymerase (NEB). ATL14 (presumed full-length provirus, 5′ and 3′LTR-host junctions detected by HTS clonality): Forward primer (5′-TGGGGCGACATCTGAAGAAA-3″: SEQ ID NO: 8) and Reverse primer (5′-TGCAGGGTTGGAGTTTCAGA-3″: SEQ ID NO: 9) produced a 750-bp (wild-type chromosome) and ˜9000 bp band including the provirus. ATL11-R-LN (lymph node, relapse): Forward primer (5′-CCTTAATCACGCTCTGGTGC-3′: SEQ ID NO: 10) and Reverse primer (5′-GCGGACTTGGGCCTTATCAT-3′: SEQ ID NO: 11) produced a 460-bp band (wild-type chromosome) and a ˜4000 bp band including presumed 5′LTR-deleted type-2 defective provirus. Libraries prepared from gel-purified PCR products (Ligation Sequencing kit 2D-R9.4) were sequenced with a SpotON Flow Cell Mk-I-R9.4 (Oxford Nanopore Technologies). FASTQ sequences were extracted using Poretools and mapped via BWA-MEM to custom genomes with provirus integrated into the appropriate sites.

### Example 14: T-Cell Receptor (TCR) Gene Rearrangement

TCR-gamma (γ) gene rearrangement was assessed using the established Euroclonality (Biomed-2) protocol (Van Dongen J J M et al.: Design and standardization of PCR primers and protocols for detection of clonal immunoglobulin and T-cell receptor gene recombinations in suspect lymphoproliferations: Report of the BIOMED-2 Concerted Action BMH4-CT98-3936. Leukemia 17:2257-2317, 2003).

### Example 15: Immuno-Phenotyping of Blood Cells

The presence of abnormal lymphocyte populations that express CD4 (clone RPA-T4), CD25 (2A3), HLA-DR (L243), CD3^{dim} (UCHT1) and CD7^{absent} (124-1D1) was examined by flow cytometry (FCM) according to Euroflow standardization protocols.

### Example 16

Clonality implies the state of a cell or a substance being derived from one source or the other. Thus there are terms like polyclonal—derived from many clones; oligoclonal—derived from a few clones; and monoclonal—derived from one clone. These terms are most commonly used in context of antibodies or immunocytes. All cells where the HTLV-1 provirus is inserted at the same site in the cellular genome represent “Sister cells”. A “clone” is a population of sister cells. Random DNA fragmentation by sonication is a key feature to allow the quantification of the abundance of a unique insertion site. For each unique insertion site the number of amplicons of different length were counted. An “amplicon” is a molecule generated during PCR.

Additionally, misreading of the tag index during the sequencing could lead to the attribution of a particular insertion site to different samples. In order to avoid this issue not only the 6 bp tag information was taken into account but also the total number of distinct shear sites and the total number of reads for a given insertion site to attribute the insertion site to the correct sample.

### Example 17: Results of HTS Mapping of Proviral Integration Sites Provides a Quantitative Measure of Molecular Response

The longitudinal clonality analysis of retrospective PBMC samples of five ATL patients diagnosed with a leukemic subtype (4 acute and 1 unfavorable chronic subtype) which all achieved clinical remission upon induction therapy was performed. This was realized by applying an optimized HTS-based method to map HTLV-1 integration sites and quantify the abundance of the corresponding clones, enabling the molecular follow-up of the predominant presumed tumor clone. Patients' and samples' characteristics are summarized in Table 1. While all five patients eventually relapsed, the duration of hematological remission, the clinical course and the survival outcome were variable between patients. Two patients achieved a protracted clinical remission of 5.8 and 2.4 years (ATL11 and ATL60 respectively; FIG. 1a, b), while three patients showed a rapid relapse with significantly shorter remission of 4.3, 5.3 and 3.7 months for ATL7, ATL14 and ATL100 respectively (Table 1, FIG. 1c-e).

For each ATL, the clonal architecture (i) at diagnosis, (ii) at relapse, and (iii) at intermediate time points was analyzed that consisted of either a single (CR1; ATL7 and ATL100) or multiple (CR1, CR2, CR3; ATL11, ATL14 and ATL60) longitudinal samples collected over the period of hematological remission. PVL (the number of proviral copies per 100 PBMCs), TCR-γ rearrangement profiles and blood cell immuno-phenotypes were also recorded for all samples. Patients' clinical and biological data during follow-up are reported in Table 2. HTS mapping of HTLV-1 integration sites at diagnosis revealed a single dominant integration site that constituted 92.75% to 99.86% (mean 95.9%) of proviral genomes in 4 of the 5 ATL cases (ATL7, ATL11, ATL14 and ATL100). In the remaining tumor (ATL60) there was evidence of 4 dominant proviruses present at the same frequency in a single malignant clone, consistent with the observation of a single TCR-γ rearrangement (total relative abundance 99.05%). All patients were treated and achieved complete clinical remission, which was characterized by normalized CBC, <5% abnormal lymphocytes and the absence of measurable tumors for >4 weeks. For ⅖ patients, molecular analysis revealed that the predominant HTLV-1 insertion site of the presumed malignant clone fell from 97.32% to 1.87% (ATL11) and from 99.05% to 2.15% (ATL60) following treatment. In both patients, the clone frequency distribution of HTLV-1 infected cells at clinical remission was composed of multiple low abundance clones, of which the unique presumed malignant integration site contributed to less than 3% of proviral genomes (FIG. 1, a-b, CR1). One of the patients (ATL11) remained in clinical and molecular remission for 5 years and 11 months with no significant change in clone frequency and modest fluctuations in PVL (CR2 and CR3; FIG. 1a), while the second patient (ATL60) showed a gradual yet moderate recurrence of the malignant clone over the 2 year and 4 month period of complete hematological remission with no increase in PVL (9.25% and 36.95% for CR2 and CR3 respectively, FIG. 1b). Clonality analysis of ATL60 at relapse revealed the full recurrence of the predominant integration sites detected at diagnosis with an overall clonal abundance of 99.46%. ATL11 relapsed with a lymphoma subtype and with a different clone. This new clone constituted 89.3% of proviral genomes. The observation of clonal change is consistent with previous reports that demonstrated the emergence of a distinct clone during the clinical course of ATL. The dominant integration site was supported by 3′LTR-host junctions yet 5′LTR dependent reads were not retrieved in the sequencing output, strongly suggesting that the new malignant clone that emerged at relapse represented a 5′LTR-deleted provirus. The present inventors verified that this was effectively the case by applying the long-range Oxford Nanopore sequencing technology to characterize the provirus and its genomic boundaries in the lymphoma that developed at relapse. This demonstrated the ability of the 5′/3′ dual HTS approach in accurately detecting 5′LTR-deleted type 2-defective proviruses (FIG. 2).

Clonality analysis of the remaining ⅗ patients after induction therapy revealed that, contrary to ATL11 and ATL60, the relative abundance of the malignant clone identified at diagnosis remained dominant at clinical remission (73.4%, 43.24% and 55.43% at CR1; 92.75%, 99.86% and 94.95% at diagnosis for ATL7, ATL14 and ATL100 respectively) while the clinical response criteria were consistent with complete hematological remission and the PVLs had decreased 1.7- to >1000-fold (Table 2 and FIG. 1, c-e). These patients relapsed after 4.3, 5.3 and 3.7 months respectively with the dominant malignant clone >86% (86.20%, 88.1%, and 99.12% respectively). Thus, the molecular follow-up of these three patients revealed refractoriness to first-line therapy at time points where clinical response criteria indicated complete hematological remission.

### Example 18: Assessing the Clone Frequency Distribution by HTS Outperforms other Currently Available Molecular Methods

TCR-γ rearrangement data were recorded as well as the immuno-phenotype profiles of blood cells measured by FCM (FIG. 1 and Table 2). These assays have been integrated in the routine assessment of hematological malignancies at the Necker hospital. While at certain time points clonally rearranged TCR-γ and/or FCM were better indicators of recurrence than the standard hematological response criteria, both these assays had their limitations. With regards to TCR-γ, HTS clonality was a superior predictor of refractoriness to induction therapy (FIG. 1, ATL100 CR1, ATL14 CR1 and CR2). Another advantage of the HTS approach is that, in contrast to the TCR-γ assay, it enabled a quantitative measure of recurrence after a short period of molecular remission (FIG. 1, ATL60) and showed higher sensitivity (FIG. 1 d, CR1, 0.007% absolute abundance versus ˜1% sensitivity for the TCR-γ assay). In the case of FCM, although it revealed an abnormal lymphocyte population that expressed CD4⁺ CD25⁺ CD7⁻ and CD3^{dim} in 4 of the 5 ATLs, the faithful interpretation of FCM data for the accurate detection of residual disease using the CD3^{dim} status remained problematic given the overlap of CD3 expression levels with the distribution observed in healthy individuals. Furthermore, not all ATL cases show CD3^{dim} phenotypes (Table 2, ATL100: CD3^{high}), demonstrating the limitations of FCM as a marker of molecular response. In conclusion, the method of the present invention involving HTS clonality is superior to any of the other assays examined today.

1. A method for preparing a linear PCR product from genomic DNA derived from cells of a host subject infected with an retrovirus or a subject suffering from a disease associated with the retrovirus,
wherein the PCR product contains a target sequence comprising an integration site of the retrovirus in the host genomic DNA of the cells, the integration site comprising at least the terminal end of 3′-LTR or 5′-LTR sequence of the retrovirus and the adjacent host genomic DNA sequence,
wherein the PCR product comprises a first terminus and a second terminus and sequences in the following order:
sequences specific for the first terminus,
a sequence comprising at least 6 consecutive random nucleotides followed by a linker sequence,
host genomic DNA sequence,
at least the terminal end of 3′-LTR or 5′-LTR sequence of the retrovirus, and sequences specific for the second terminus;
a) isolating genomic DNA from cells derived from the subject;
b) shearing the genomic DNA obtained in step a);
c) carrying out an extension reaction using the sheared DNA obtained in step b) in the presence of a primer binding to 5′-LTR of the retrovirus, a primer binding to 3′-LTR of the retrovirus, dNTPs and a DNA polymerase, wherein the dNTPs comprise labelled dNTPs and/or wherein the primers are labelled, thereby producing a double stranded DNA product comprising a labelled DNA strand having a 3′ single nucleotide overhang; wherein the label represents a binding ligand;
d) ligating a linker having a 3′ single nucleotide overhang to the terminus of the product obtained in step c), wherein the 3′ single nucleotide overhang of the linker is complementary to the 3′ single nucleotide overhang of the labelled DNA strand; thereby introducing the sequence comprising at least 6 consecutive random nucleotides followed by a linker sequence and at least one of the sequences specific for the first terminus,
e) isolating the product obtained in step d) using a receptor binding to the binding ligand;
f) carrying out at least one PCR reaction in the presence of the product obtained in step e) as template, using a first primer specific the first terminus and a second primer specific the second terminus of the product obtained in step e), wherein the first and the second primer each comprise at least one additional sequence different from each other at its 5′-end, thereby producing the linear PCR product comprising the additional sequence specific for the first terminus and the additional sequence specific for the second terminus.

2. The method according to claim 1, wherein the DNA polymerase used in the extension step c) adds a single extra nucleotide to the 3′-end of the synthetized DNA strand.

3. The method according to claim 1, wherein in step c) a double stranded DNA product is obtained comprising at least the terminal end of 3′-LTR or 5′-LTR sequence of the retrovirus and the adjacent host genomic DNA sequence of the insertion site as well as the shear site.

4. The method according to claim 1, wherein in step d) the linker is added to that terminus of the product obtained in step c) which is opposite to the terminus carrying the 3′-LTR or 5′-LTR, respectively.

5. The method according to claim 1, wherein in step d) a double stranded DNA comprising a labelled DNA strand is obtained, wherein the double stranded DNA product comprises at least the terminal end of 3′-LTR or 5′-LTR sequence of the retrovirus, the adjacent host genomic DNA sequence of the insertion site as well as the shear site, a sequence comprising at least 6 consecutive random nucleotides followed by a linker sequence and at least one of the sequences specific for the first terminus.

6. The method according to claim 1, wherein in step d) a linker having an overhanging single nucleotide is ligated to that terminus of the product obtained in step c) having the 3′ overhanging single nucleotide,
wherein the overhanging single nucleotide of the linker hybridizes to the 3′ overhanging single nucleotide of the DNA strand synthesized in step c); thereby producing a double stranded DNA product comprising a labelled DNA strand, and
wherein the double stranded DNA product comprises at least the terminal end of 3′-LTR or 5′-LTR sequence of the retrovirus and the adjacent host genomic DNA sequence of the insertion site as well as the shear site, a sequence comprising at least 6 consecutive random nucleotides followed by a linker sequence and at least one of the sequences specific for the first terminus

7. The method according to claim 1, wherein the overhanging single nucleotide added to the 3′-end of the synthetized DNA strand in step c) is deoxyadenosine and the overhanging single nucleotide of the linker added in step d) is desoxythymidine.

8. The method according to claim 1, wherein the PCR product comprises in the following order
X4 sequence,
X3 sequence,
a tag sequence comprising 6 to 30 nucleotides,
host genomic DNA sequence,
at least the terminal end of 3′-LTR or 5′-LTR sequence of the retrovirus,
X1 sequence, and
X2 sequence;
and wherein step f) comprises the following steps:
f1) carrying out a first PCR reaction in the presence of the product obtained in step e) as template, a primer binding to the X3 sequence, a primer binding to 5′-LTR, a primer binding to 3′-LTR, wherein the primer binding to 5′-LTR and 3′-LTR, respectively, comprise an additional X1 sequence at their 5′-ends, thereby producing a PCR product comprising also the X1 sequence;
f2) carrying out a second PCR reaction in the presence of the product obtained in step f1) as template, a primer binding to the X3 sequence having an additional X4 sequence at its 5′-end, a primer binding to the X1 sequence having an additional X2 sequence at its 5′-end, thereby producing a PCR product comprising also the X2 sequence and the X4 sequence.

9. The method according to claim 1, wherein the binding ligand and the receptor are selected from the group of binding pairs consisting of biotin/avidin, biotin/streptavidin; digoxygenin/anti-digoxygenin antibody;
hapten/anti-hapten antibody; and antigen/antibody.

10. The method according to claim 1, wherein the retrovirus is HTLV-1, the HTLV-1-associated disease is Adult T-cell leukemia/lymphoma (ATL), and the genomic DNA is derived from peripheral blood mononuclear cells (PBMCs).

11. A method for determining and longitudinally monitor the dominant leukemic T lymphocyte clone in subjects suffering from Adult T-cell leukemia/lymphoma (ATL), the method comprising:
preparing a linear PCR product by the method according to claim 1,
subjecting the linear PCR product to multiplex sequencing thereby determining all insertion sites and all shearing sites, wherein the shearing sites are correlated to the respective insertion site,
counting the number of different shear sites for each insertion site representing a specific T lymphocyte clone,
removing any PCR duplicate from consideration by eliminating reads that have the same insertion site and the same random tag, and
determining the abundance of each specific T lymphocyte clone therefrom.

12. The method according to claim 11, further comprising judging, on the basis of the abundance of each specific T lymphocyte clone, the likelihood of recurrence of Adult T-cell leukemia/lymphoma (ATL).

13. The method according to claim 11, further comprising judging, on the basis of the abundance of each specific T lymphocyte clone, the success of treatment of Adult T-cell leukemia/lymphoma (ATL).

14. The method according to claim 11,
wherein a higher abundance of a specific T lymphocyte clone indicates a higher likelihood of recurrence of Adult T-cell leukemia/lymphoma (ATL) and/or that the treatment of Adult T-cell leukemia/lymphoma (ATL) is not successful; and
wherein a lower abundance of a specific T lymphocyte clone indicates a lower likelihood of recurrence of Adult T-cell leukemia/lymphoma (ATL) and/or the that the treatment of Adult T-cell leukemia/lymphoma (ATL) is successful.

15. The method according to claim 11,
wherein the specific T lymphocyte clone is the same as identified as dominant leukemic clone at diagnosis.
